(19) United States
(12) Patent Application Publication
(10) Pub. No.: US 2026/0117281 A1
(43) Pub. Date: Apr. 30, 2026

(54) COMPARATIVE ASSESSMENT OF NANOPARTICLE DELIVERY VEHICLES

(71) Applicant: NUTCRACKER THERAPEUTICS, INC., Boston, MA (US)

(72) Inventors: Pei-Ken HSU, San Francisco, CA (US); Sangeeta NATH, Freemont, CA (US)

(21) Appl. No.: 19/420,940

(22) Filed: Dec. 16, 2025

Related U.S. Application Data
(63) Continuation of application No. PCT/US2024/033973, filed on Jun. 14, 2024.
(60) Provisional application No. 63/521,480, filed on Jun. 16, 2023.

(51) Int. Cl. C12Q 1/6809; C12N 9/22; C12N 15/11; C12Q 1/34; C12Q 1/6806

(52) U.S. Cl. CPC C12Q 1/6809; C12N 9/226; C12N 15/111; C12Q 1/34; C12Q 1/6806; C12N 2310/20; C12Q 2600/136; G01N 2333/922

(57) ABSTRACT

Provided herein are compositions and methods for identifying effective delivery vehicles (DVs) for mRNA delivery to and expression in specific cells or tissues in culture (in vitro) or in live animals (in vivo) from a pool of co-administered DVs.

### CROSS REFERENCE TO RELATED APPLICATIONS

This application is a continuation of, and claims the benefit of and priority to, International Application No. PCT/US2024/033973, filed Jun. 14, 2024, which claims priority to U.S. Provisional 62/521,480, filed on Jun. 16, 2023, each of which is incorporated by reference herein in their entirety.

### FIELD

The disclosed subject matter relates generally to nanoparticle technology and more specifically to the field of comparative assessments of nanoparticle delivery vehicles.

### BACKGROUND

The application of lipid nanoparticles (LNP) and LNP-like delivery vehicles (DVs) to the delivery of mRNA cargo has greatly expanded the capability of targeting specific tissues at high efficiency for gene and cell therapies. By the delivery and subsequent expression of cargo mRNAs, cell function and behavior can be modified to produce therapeutic effects. This is especially valuable for in vivo gene therapy, for which viral delivery is the other main delivery approach. There are multiple complications with viral delivery approaches that hamper the broad and safe use of this method in the clinic, however.

One of the main challenges in the use of DVs for gene therapy is the lack of efficient methods to empirically identify the most effective DV composition, from a pool of DVs, that mediates good delivery and expression of the cargos in specific cell types or tissues in live animals, or even in cell cultures. It is prohibitively expensive to assess the efficacy of each DV composition individually.

High-throughput (HTP) methods have been designed to screen and identify the most desirable DV from a pool of DVs co-administered into the animals. Existing HTP methods for DV screening rely on co-encapsulating a unique short DNA barcode in each specific DV (Dahlman, 2017). By reading out the relative frequency of each DNA barcode from the collected cells or tissue samples, the DVs that mediate higher delivery efficiency may be identified, for example by using Next Generation Sequencing (NGS)-based digital counting methods. This approach, however, cannot differentiate between successful cellular uptake of the DV and successful expression of the cargo, with the latter function requiring the release of the mRNAs into the cell cytoplasm where they can become available for ribosome-mediated translation. Therefore, a HTP method that can identify DV compositions that mediate effective mRNA delivery and expression is needed to facilitate the successful development of mRNA gene therapies.

Komor (2016) disclose the development of ‘base editing’, an approach to genome editing that can enable the direct, irreversible conversion of one target DNA base into another in a programmable manner, without requiring dsDNA backbone cleavage or a donor template, by engineering fusions of CRISPR/Cas9 and a cytidine deaminase enzyme that retain the ability to be programmed with a guide RNA, do not induce dsDNA breaks, and mediate the direct conversion of cytidine to uridine, thereby effecting a C→T (or G→A) substitution.

Gaudelli (2017) disclose adenine base editors (ABEs) that mediate the conversion of A⋅T to G⋅C in genomic DNA, and the evolution of a transfer RNA adenosine deaminase to operate on DNA when fused to a catalytically impaired CRISPR-Cas9 mutant, as well as directed evolution and protein engineering resulting in seventh-generation ABEs that convert targeted A⋅T base pairs efficiently to G⋅C.

Anzalone (2019) disclose prime editing, a genome editing method that directly writes new genetic information into a specified DNA site using a catalytically impaired Cas9 endonuclease fused to an engineered reverse transcriptase, programmed with a prime editing guide RNA (pegRNA) that both specifies the target site and encodes the desired edit.

### SUMMARY

The disclosure provides versatile methods for assessing the efficacy of delivery vehicle of cargoes to cells or tissues of any type. The versatility of the methods is apparent in realizing that the methods of the disclosure allow for the rapid parallel assessment of large quantities of differently formulated or configured delivery vehicles, including lipid nanoparticle and lipid nanoparticle-like delivery vehicles. Moreover, the methods of the disclosure can be performed in vitro using ex vivo cell samples or cell cultures, or in vivo. The result of practicing the disclosed methods is the rapid, cost-effective identification of effective and particularly suitable delivery vehicles for any of the many varieties of delivery vehicle cargoes and for any of the many target cell types or tissues.

In one aspect, the disclosure provides a method of assessing the targeting efficacy of a delivery vehicle comprising: (a) contacting each of at least two cell types with a delivery vehicle, wherein the delivery vehicle comprises a coding region for a Cas9 protein or a Base Editor protein and an single guide RNA (sgRNA) comprising a targeting sequence complementary to a portion of the genome of at least one of the two cell types; (b) maintaining the cell types under conditions suitable for: (i) expressing the coding region for the Cas9 protein or the Base Editor protein (2,3); (ii) forming a Cas9-sgRNA or a BE-sgRNA ribonucleoprotein; and (iii) modifying the genome of the at least two cell types; (c) measuring a level of genome modification in the at least two cell types; and (d) identifying the delivery vehicle as effectively targeting the target cell type if the level of genome modification is greater in the target cell type than in at least one other cell type. In some embodiments, the coding region encodes either a Cas9 protein or a Base Editor protein. In some embodiments, the targeting sequence is complementary to the genome of each of the at least two cell types, including embodiments in which the target sequence is perfectly complementary to the genome of each of the at least two cell types. In some embodiments, the target sequence is complementary to a portion of the euchromatin of each of the at least two cell types, including embodiments wherein the target sequence is perfectly complementary to a portion of the euchromatin of each of the at least two cell types.

Another aspect of the disclosure is drawn to a method of assessing the target efficacy of a delivery vehicle comprising: (a) contacting cells of a cell type with a plurality of delivery vehicles, wherein each delivery vehicle comprises a coding region for a Cas9 protein or a Base Editor protein and an sgRNA comprising a targeting sequence, wherein each delivery vehicle comprises the coding region for at least one gene-editing reagent and an sgRNA comprising a unique targeting sequence complementary to a portion of the genome of the cell type; (b) maintaining the cells under conditions suitable for: (i) expressing the coding region for the at least one gene-editing reagent; (ii) forming a Cas9-sgRNA or a BE-sgRNA ribonucleoprotein; and (iii) modifying the genome of the cells; (c) measuring locations and levels of genome modifications in the cells; and (d) identifying the delivery vehicle as effectively targeting a cell type if the level of genome modification is greater in the cell type compared to the level of genome modification resulting from use of at least one other delivery vehicle. In some embodiments, the coding region encodes either a Cas9 protein or a Base Editor protein (2,3). In some embodiments, the targeting sequence is complementary to the genome of each of the at least two cell types, including embodiments wherein the target sequence is perfectly complementary to the genome of each of the at least two cell types. In some embodiments, the target sequence is complementary to a portion of the euchromatin of each of the at least two cell types, including embodiments wherein the target sequence is perfectly complementary to a portion of the euchromatin of each of the at least two cell types. In some embodiments, the method further comprises administering the targeting delivery vehicle to a subject in need.

Yet another aspect of the disclosure is directed to a method of assessing the target efficacy of a delivery vehicle comprising: (a) contacting cells of a cell type with a plurality of delivery vehicles, wherein each delivery vehicle comprises a coding region for a Prime Editor protein (4) and a Prime Editor guide RNA (pegRNA) (4) comprising a barcode sequence, wherein the barcode sequence in the pegRNA in each of the delivery vehicles is unique; (b) maintaining the cells under conditions suitable for: (i) expressing the coding region for the Prime Editor protein; (ii) forming a PE-pegRNA ribonucleoprotein; and (iii) modifying the genome (insertion of the associated barcode) of the cells; (c) measuring the levels of genome modifications in the cells; and (d) identifying the delivery vehicle as effectively targeting a cell type if the level of genome modification resulting from use of the delivery vehicle is greater than the level of genome modification resulting from use of at least one other delivery vehicle. In some embodiments, the targeting sequence is complementary to the genome of each of the at least two cell types, including embodiments wherein the target sequence is perfectly complementary to the genome of each of the at least two cell types. In some embodiments, the target sequence is complementary to a portion of the euchromatin of each of the at least two cell types, including embodiments wherein the target sequence is perfectly complementary to a portion of the euchromatin of each of the at least two cell types. In some embodiments, the method further comprises administering the targeting delivery vehicle to a subject in need. In some embodiments, the unique barcode sequence is at least 2 nucleotides (generally>=6 nucleotides).

Other features and advantages of the disclosure will become apparent from the following detailed description, including the drawing. It should be understood, however, that the detailed description and the specific examples, while indicating embodiments, are provided for illustration only, because various changes and modifications within the spirit and scope of the disclosure will become apparent to those skilled in the art from the detailed description.

### BRIEF DESCRIPTION OF THE DRAWING

FIG. 1 shows a pooled DV screen with DVs comprising co-formulated cargos of a plurality of unique single guide RNAs (i.e., sgRNAs) and mRNAs encoding either Cas9 or Base Editor (i.e., BE). The percentage of genomic edits (insertions/deletions (i.e., indels) by Cas9 or base changes by BE) at each unique sgRNA-specified location provides a measure of delivery success, by the associated DV, in terms of targeting the desired cell type(s) and/or tissue(s) and in terms of the expression of the mRNA encoding either Cas9 or BE.

FIG. 2 shows a pooled DV screen with Prime Editor mRNAs (i.e., PE mRNAs) and Prime Editor gRNAs (i.e., pegRNAs) comprising barcodes as co-formulated cargos. As noted herein, the pegRNAs are unique as a result of the unique barcode sequences that can be incorporated (reverse transcribed) into a defined location of the genome. The unique barcodes incorporated distinguish the successful functional delivery by the associated DVs. The percentage of each inserted barcode at a specific genomic location provides a measure of delivery success by the associated DV.

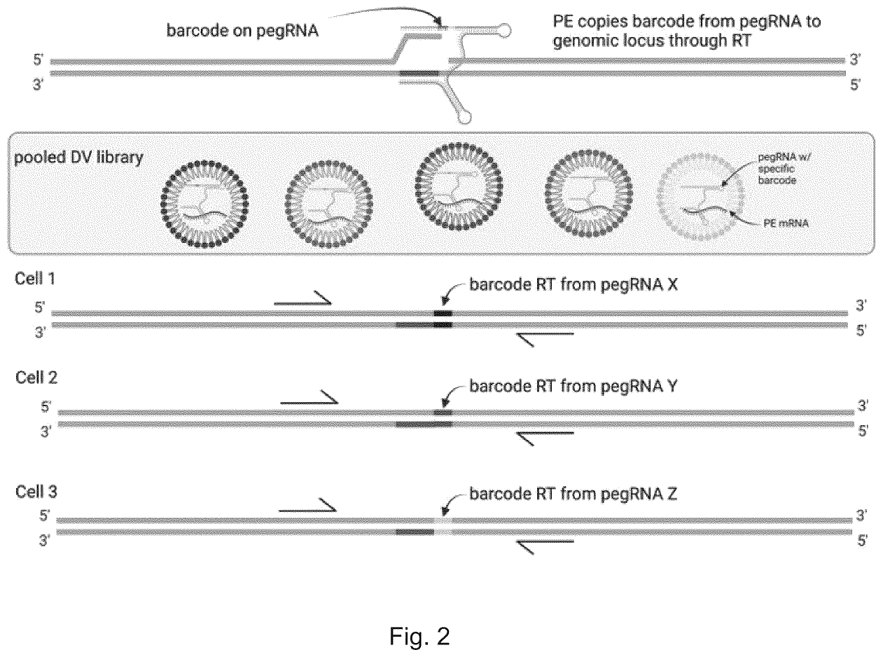

### DETAILED DESCRIPTION

The disclosure provides compositions and methods for conducting comparative assays to identify effective mRNA delivery vehicles (DVs) for mRNA delivery targeting expression in specific cells or tissues either in vitro (e.g., in culture) or in vivo (e.g., in live animals, such as humans), where the effective DVs are identified in a pool of co-administered DVs. The comparative assays assess both physical targeting of the DVs to particular cell(s) and/or tissue(s) and expression of the mRNA cargo delivered by the DVS.

The disclosed methods are based on encapsulating unique CRISPR guide RNAs (e.g., single guide RNAs (i.e., sgRNAs) or Prime Editor guide RNAs (i.e., pegRNAs)) comprising barcodes into specific, distinct DVs. mRNA encoding Cas9 effectors (Cas9, Base Editor, or Prime Editor) are also encapsulated along with the CRISPR guide RNAs in every DV. Successful delivery and expression of the RNA cargos result in the binding of expressed Cas effector protein to the co-delivered CRISPR guide RNAs to form Cas9 effector ribonucleoproteins (i.e., RNPs). Cas effector RNPs generate specific genomic modifications according to the sequence of CRISPR guide RNAs. Quantitative readout by sequencing and determination of the level of specific genomic modification by each unique CRISPR guide RNAs reflects the efficiency of associated DVs in the delivery and expression of the cargo mRNA. These methods can be applied in cell cultures of interest (i.e., in vitro) or in live animals (i.e., in vivo) to identify effective delivery vehicle compositions by screening a pool of DVS. Effective DVs mediate successful delivery and expression of mRNA cargos in specific cell type(s) and/or tissue(s).

In the methods disclosed herein, the methods produce readouts of permanent genomic modifications at one or more phenotypically neutral loci in the genome. These genomic modifications can be detected by sequencing in any cell or tissue of interest. Because each DV can be associated with a different guide RNA, resulting in different yet predictable genomic modifications, this method is highly amenable to multiplexing, as each genomic edit identified by sequencing can be associated with a unique RNA guide.

To generate genomic modifications useful in the comparative assessment of DV effectiveness according to the disclosure, the following steps are executed: (i) co-delivery of both the Cas effector mRNA and a guide RNA into cells, (ii) translation of the Cas effector (e.g., Cas9, base editor [BE]^{2,3}, or prime editor [PE]⁴) mRNA into protein, (iii) formation of a Cas9 effector-guide RNA ribonucleoprotein (i.e., RNP) complex in the cytoplasm and (iv) translocation of the RNP complex into the nucleus where genome editing can take place. By linking expression of a gene (Cas effector mRNA) to a quantifiable and permanent modification in the genome, a high number of DV compositions can be pooled and interrogated for function in an empirical manner allowing for the selection of optimal DV compositions for different therapeutic modalities.

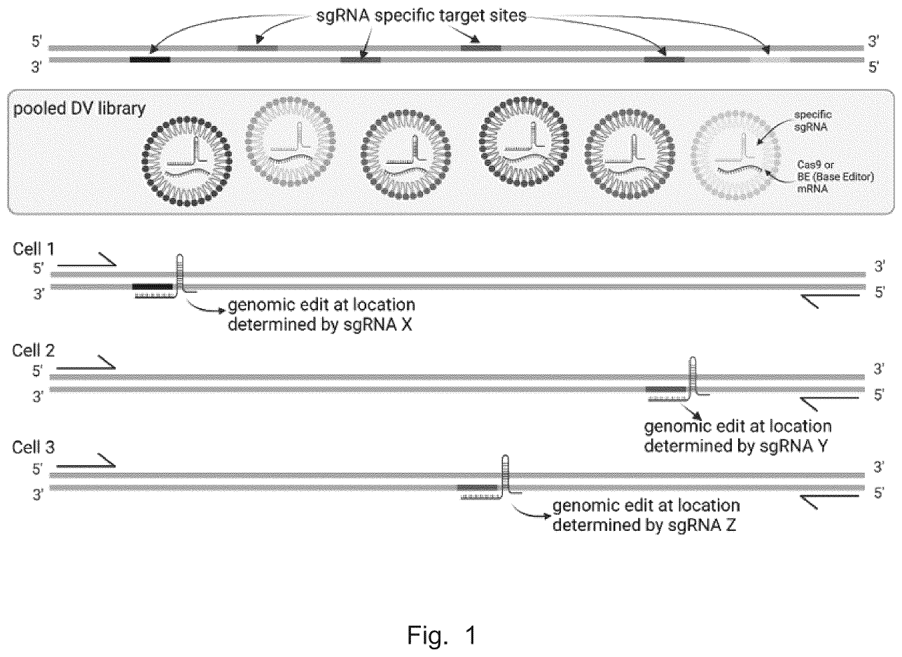

Two aspects of the disclosure are drawn to different methods for the comparative assessment of DV effectiveness. These methods are schematically illustrated in the figures. Methods according to the first aspect of the disclosure are disclosed in principle in FIG. 1. These methods use the specific location of genomic edits as an identifier for each unique DV. Single guide RNAs (sgRNAs) are co-formulated with Cas9 or BE mRNAs as DV cargos, with each unique sgRNA encapsulated by, and associated with, a unique DV. In this first aspect, each of the sgRNAs in the pool of DVs being assessed comprises a sequence complementary (e.g., perfectly complementary) to a different region of the genome of the target cell type. Successful delivery and expression of the cargos results in genomic edits in the form of small insertions or deletions (indels) or base changes (A to G or C to T) depending on whether Cas9 or BE is used. When Cas9 mRNAs are delivered and expressed, Cas9-sgRNA RNPs generate double-strand breaks (DSBs) in the genome at a location determined by the target, or seed, sequence of the sgRNA. This event is followed by imprecise repair of DSBs by non-homologous end joining (NHEJ), resulting in the formation of indels, as is known in the art. In contrast, when BE mRNAs are delivered and expressed, BE-sgRNA RNPs instruct the base change (A to G or C to T) at the location determined by the target (i.e., seed) sequence of the sgRNA. Because the location of the genomic edits is solely determined by the sgRNA target (i.e., seed) sequence, we can identify the DV whose successful delivery into the cells results in the specific genomic modification, as illustrated in FIG. 1.

In the methods according to the second aspect of the disclosure, each pegRNA comprises a common targeting sequence complementary (e.g., perfectly complementary) to a specific genomic locus and a unique barcode sequence. Each DV in the pool of DVs being assessed contains a pegRNA with the common targeting sequence and a unique barcode sequence that identifies the particular DV. Methods according to this aspect result in the insertion of an unique barcode sequence of the pegRNA at one gemonic locus identifying a particular DV. This method leverages prime editing in which PE, composed of a Cas9 nickase fused to a reverse transcriptase, generates a nick in the genome and then inserts the barcode sequence through reverse transcription (i.e., RT) at the nick site. A second component of prime editing, the prime editing guide RNA (i.e., pegRNA), instructs the PE to nick at a defined genomic location and serves as a RT template for a specific barcode. PE mRNAs and pegRNAs are co-formulated as cargos, with each unique barcode-containing pegRNA encapsulated by, and associated with, a unique DV. Successful delivery and expression of the cargo results in prime editing-mediated insertion of a specific barcode sequence in a defined genomic location. By comparing the frequency of specific barcodes inserted at the defined genomic location, the relative delivery efficiencies of the associated DVs can be determined.

### References

1. Dahlman JE, Kauffman KJ, Xing Y, Shaw TE, Mir FF, Dlott CC, Langer R, Anderson DG, Wang ET: Barcoded nanoparticles for high throughput in vivo discovery of targeted therapeutics. Proc Natl Acad Sci USA 2017, 114:2060-2065.
2. Komor AC, Kim YB, Packer MS, Zuris JA, Liu DR: Programmable editing of a target base in genomic DNA without double-stranded DNA cleavage. Nature 2016, 533:420
3. Gaudelli NM, Komor AC, Rees HA, Packer MS, Badran AH, Bryson DI, Liu DR:

Programmable base editing of A⋅T to G⋅C in genomic DNA without DNA cleavage. Nature 2017, 551:464-471.
4. Anzalone AV, Randolph PB, Davis JR, Sousa AA, Koblan LW, Levy JM, Chen PJ, Wilson C, Newby GA, Raguram A, Liu DR: Search-and-replace genome editing without double-strand breaks or donor DNA. Nature 2019, 576:149-157.

All patents and other publications identified are expressly incorporated herein by reference in their entireties or in relevant part, as would be apparent to the skilled person from the context, for the purpose of describing and disclosing, for example, the methodologies described in such publications that might be used in connection with information described herein.

What is claimed is:

1. A method of assessing targeting efficacy of a delivery vehicle comprising:
(a) contacting each of at least two cell types with the delivery vehicle, wherein the delivery vehicle comprises a coding region for a Cas9 protein or a Base Editor protein and an sgRNA comprising a targeting sequence complementary to a portion of a genome of at least one of the at least two cell types;
(b) maintaining the at least two cell types under conditions suitable for:
(i) expressing the coding region for the Cas9 protein or the Base Editor protein;
(ii) forming a Cas9-sgRNA or a BE-sgRNA ribonucleoprotein; and
(iii) modifying the genome of the at least two cell types;
(c) measuring a level of genome modification in the at least two cell types; and
(d) identifying the delivery vehicle as effectively targeting the target cell type if the level of genome modification is greater in the target cell type than in at least one other cell type.

2. The method of claim 1, wherein the coding region encodes the Cas9 protein.

3. The method of claim 1, wherein the coding region encodes the Base Editor protein.

4. The method of claim 1, wherein the targeting sequence is complementary to the genome of each of the at least two cell types.

5. The method of claim 4, wherein the targeting sequence is perfectly complementary to the genome of each of the at least two cell types.

6. The method of claim 1, wherein the targeting sequence is complementary to a portion of a euchromatin of each of the at least two cell types.

7. The method of claim 1, wherein the targeting sequence is perfectly complementary to a portion of a euchromatin of each of the at least two cell types.

8. The method of claim 1, further comprising administering the delivery vehicle to a subject in need.

9. A method of assessing target efficacy of a delivery vehicle comprising:
(a) contacting cells of a cell type with a plurality of delivery vehicles, wherein each delivery vehicle comprises a coding region for a Cas9 protein or a Base Editor protein and an sgRNA comprising a targeting sequence, wherein each delivery vehicle comprises a coding region for at least one gene-editing reagent and an sgRNA comprising a unique targeting sequence complementary to a portion of a genome of the cell type;
(b) maintaining the cells under conditions suitable for:
(i) expressing the coding region for the at least one gene-editing reagent;
(ii) forming a Cas9-sgRNA or a BE-sgRNA ribonucleoprotein; and
(iii) modifying the genome of the cells;
(c) measuring locations and levels of genome modifications in the cells; and
(d) identifying the delivery vehicle as effectively targeting a cell type if the level of genome modification is greater in the cell type compared to the level of genome modification resulting from use of at least one other delivery vehicle.

10. The method of claim 9, wherein the coding region encodes the Cas9 protein.

11. The method of claim 9, wherein the coding region encodes the Base Editor protein.

12. The method of claim 9, wherein the targeting sequence is complementary to the genome of each of the at least two cell types.

13. A method of assessing target efficacy of a delivery vehicle comprising:
(a) contacting cells of a cell type with a plurality of delivery vehicles, wherein each delivery vehicle comprises a coding region for a Prime Editor protein and a pegRNA comprising a barcode sequence, wherein the barcode sequence in the pegRNA in each of the delivery vehicles is a unique barcode sequence;
(b) maintaining the cells under conditions suitable for:
(i) expressing the coding region for the Prime Editor protein;
(ii) forming a PE-pegRNA ribonucleoprotein; and
(iii) modifying a genome of the cells;
(c) measuring the levels of genome modifications in the cells; and
(d) identifying the delivery vehicle as effectively targeting a cell type if the level of genome modification resulting from use of the delivery vehicle is greater than the level of genome modification resulting from use of at least one other delivery vehicle.

14. The method of claim 13, wherein the barcode sequence is complementary to a genome of each of the at least two cell types.

15. The method of claim 14, wherein the barcode sequence is perfectly complementary to the genome of each of the at least two cell types.

16. The method of claim 13, wherein the barcode sequence is complementary to a portion of a euchromatin of each of the at least two cell types.

17. The method of claim 13, wherein the barcode sequence is perfectly complementary to a portion of a euchromatin of each of the at least two cell types.

18. The method of claim 13, further comprising administering the delivery vehicle to a subject in need.

19. The method of claim 13, wherein the unique barcode sequence is at least 2 nucleotides.

20. The method of claim 13, wherein the unique barcode sequence is at least 6 nucleotides.
